(19) United States
(12) Patent Application Publication
(10) Pub. No.: US 2024/0044787 A1
(43) Pub. Date: Feb. 8, 2024

(54) CORONAVIRUS MUTATION DETERMINATION DEVICE COMPRISING METAMATERIAL ARRAY AND ELECTROMAGNETIC WAVE IRRADIATION UNIT

(71) Applicant: KOREA INSTITUTE OF SCIENCE AND TECHNOLOGY, Seoul (KR)

(72) Inventors: Minah SEO, Seoul (KR); Hyun Seok SONG, Seoul (KR); Hyun Gyu SONG, Seoul (KR); Jeongmin JANG, Seoul (KR); Yeonkyung LEE, Seoul (KR); Soohyun LEE, Seongnam-si (KR)

(21) Appl. No.: 18/365,770

(22) Filed: Aug. 4, 2023

(30) Foreign Application Priority Data
Aug. 5, 2022 (KR) 10-2022-0097738

(51) Int. Cl. G01N 21/3581

(52) U.S. Cl. CPC G01N 21/3581

(57) ABSTRACT

The present specification relates to a device and method capable of determining a coronavirus mutation using a highly sensitive label-free terahertz electromagnetic wave metamaterial sensor, and a device for diagnosing a coronavirus disease-19, amd the device can specify the amino acids of a virus belonging to the genus coronavirus having an excellent absorption in a terahertz band, and the metamaterial can amplify the corresponding signal, and the problem of coffee ring formation, which hinders the measurement and analysis in the conventional measurement and analysis of solution-based samples, can be solved by the method in which a sample containing coronavirus is applied to the surface of a metamaterial, and the optical constant value of the sample is analyzed, so that it is possible to more quickly and accurately determine the coronavirus mutation, and furthermore, there is an excellent effect in diagnosing a coronavirus disease-19.

### CROSS REFERENCE TO RELATED APPLICATION

The present application claims priority to Korean Patent Application No. 10-2022-0097738, filed Aug. 5, 2022, the entire contents of which is incorporated herein for all purposes by this reference.

### BACKGROUND OF THE INVENTION

### Field of the Invention

The present specification relates to a device and method capable of determining a coronavirus mutation using a highly sensitive label-free terahertz electromagnetic wave metamaterial sensor, and a device for diagnosing a coronavirus disease-19.

### Description about National Support Research and Development

This study is made by the support of the Korea Institute of Science and Technology Research operation cost (main project cost) of the Korea Institute of Science and Technology under the supervision of the Korea Institute of Science and Technology, and the subject name thereof is Intelligent Solution Technology for Disaster Safety (Subject Identification No.: 2E31600). This study is made by the support of Global frontier (R&D) of the Korea Institute of Science and Technology under the supervision of the National Research Foundation of Korea, and the subject name thereof is Terahertz metamaterial-based highly sensitive cancer tissue detection system (Subject Identification No.: 2019M3A6B3030638). This study is made by the support of Personal basic research (Ministry of Science and ICT) (R&D) of the Korea Institute of Science and Technology under the supervision of the National Research Foundation of Korea, and the subject name thereof is Terahertz Molecular Sensor Active Control Technology (Subject Identification No.: 2020R1A2C2007077). This study is made by the support of Personal Basic Research (Ministry of Science and ICT) of the Korea Institute of Science and Technology under the supervision of the National Research Foundation of Korea, and the subject name thereof is Terahertz Molecular Sensor Active Control Technology (Subject Identification No.: 2020R1A2C2007077). This study also is made by the support of Global frontier of the Korea Institute of Science and Technology under the supervision of the National Research Foundation of Korea, and the subject name thereof is Terahertz metamaterial-based highly sensitive cancer tissue detection system (Subject Identification No.: 2019M3A6B3030638).

### Description of the Related Art

Recently, the emergence of highly contagious and persistent mutations of coronavirus disease-19 (SARS-CoV-2 or COVID-19), as well as asymptomatic infection, has made it difficult for medical staff and quarantine authorities to respond to the coronavirus disease-19. Accordingly, various studies are being conducted to reduce the burden of the medical field by early blocking and detecting the spread of infectious diseases.

Currently, there are a PCR test and a rapid antigen test as methods for determining SARS-CoV-2, which is the cause of coronavirus disease-19 (COVID-19). The PCR test has high accuracy, but it takes a long test time of 4 hours or more, and the rapid antigen test has a short detection time of less than 20 minutes, but has a limitation in that the accuracy is low because it is difficult to distinguish between samples having similar protein structures. Viruses belonging to the betacoronavirus genus, which is one of the four genera of the coronavirus family that includes SARS-CoV-2, have more than 82% similar amino acid sequences, so there is an urgent need to develop a diagnostic technology that can quickly determnine these viruses.

On the other hand, among the spectroscopy methods sensitive to the natural vibration of biomolecules, low-frequency terahertz electromagnetic waves can also analyze information about hydrogen bonds and intermolecular forces in materials, enabling label-free biodiagnosis and characterization. Therefore, the terahertz electromagnetic waves have the potential to interpret even minute unique information and differences of biological samples such as DNA, amino acids, and monomers. However, due to a low signal-to-noise ratio, lack of signal amplification technology, and cryogenic measurement environment, it is very difficult to observe minute changes in biological samples at room temperature.

Accordingly, the present inventors attempted to develop a biodiagnostic technology by introducing a highly sensitive metamaterial capable of focusing electromagnetic waves in a specific band in order to overcome this problem. The study was conducted on the assumption that this terahertz analysis technology could be applied to discriminate between SARS-CoV-2 and mutant viruses derived from it, which do not show significant differences in the basic protein structure.

### SUMMARY OF THE INVENTION

The present invention has developed a biomolecular diagnostic platform capable of sensitively measuring a terahertz light signal reflecting the unique information of a sample even in a small amount by using a metamaterial that amplifies a signal of a specific band of electromagnetic waves. Specifically, after specifying an amino acid having an excellent absorption rate in the terahertz band, a terahertz metamaterial to amplify the corresponding signal was developed. In addition, in order to closely observe the terahertz signal change, a sample is uniformly dispersed on the surface of the metamaterial and then the optical constant value of the sample is analyzed, thereby determining monomers within a few minutes and estimating sample characteristics such as charge, polarity, and hydrophobicity index and quantities, and it was confirmed that it could be used to specify mutant viruses having similar structures by detecting changes in amino acid units.

In one aspect, an object of the present invention is to provide a device fordetermining a coronavirus mutation, including a metamaterial array that amplifies some amino acids constituting a coronavirus (Cov), and an electromagnetic wave irradiation unit that irradiates terahertz electromagnetic wave toward the metamaterial array.

In another aspect, an object of the present invention is to provide a method for determining a coronavirus mutation using the device for determining a coronavirus mutation.

In another aspect, an object of the present invention is to provide a device for diagnosing a coronavirus disease-19 (SARS-CoV-2), including a metamaterial array that amplifies some amino acids constituting the coronavirus and an electromagnetic wave irradiation unit that irradiates terahertz electromagnetic wave toward the metamaterial array, wherein a biological sample separated from an object is uniformly applied to a surface of the metamaterial.

In one aspect, the present invention provides a device for determining a coronavirus mutation, including a metamaterial array that includes a metamaterial and amplifies electromagnetic wave of a specific frequency; and an electromagnetic wave irradiation unit that irradiates terahertz electromagnetic wave toward the metamaterial array, wherein the metamaterial array amplifies some amino acids constituting a coronavirus (Cov).

In another aspect, the present invention provides a method for determining a coronavirus mutation, including the steps of applying a sample containing a coronavirus to a surface of the metamaterial of the device; and drying the metamaterial array including the metamaterial to which the sample is applied.

In another aspect, the present invention provides a device for diagnosing a coronavirus disease-19, including a metamaterial array that includes a metamaterial and amplifies electromagnetic wave of a specific frequency; and an electromagnetic wave irradiation unit that irradiates terahertz electromagnetic wave toward the metamaterial array, wherein the metamaterial array amplifies some amino acids constituting a coronavirus, and a biological sample separated from an object is uniformly applied to a surface of the metamaterial.

The device for determining a coronavirus mutation according to one aspect of the present invention can specify the amino acids of a virus belonging to the genus coronavirus having an excellent absorption rate in a terahertz band, and the metamaterial according to one aspect of the present invention can amplify the corresponding signal. Also, the problem of coffee ring formation, which hinders the measurement and analysis in the conventional measurement and analysis of solution-based samples, can be solved by a method according to one aspect in which a sample containing coronavirus is applied to the surface of a metamaterial. Accordingly, the optical constant value of the sample is analyzed, so that it is possible to more quickly and accurately determine the coronavirus mutation, and furthermore, there is an excellent effect in diagnosing a coronavirus disease-19.

### BRIEF DESCRIPTION OF THE DRAWINGS

FIG. 1 is a block diagram of a device for determining a coronavirus mutation according to an aspect of the present invention.

FIG. 2 is a diagram of a metamaterial array according to an embodiment of the present invention.

FIG. 3A, FIG. 3B, FIG. 3C, FIG. 3D, FIG. 3E and FIG. 3F are diagrams taken when a solution-based sample is applied to a metamaterial surface of a device for determining a coronavirus mutation or a device for diagnosing a coronavirus disease-19 according to an embodiment of the present invention. Specifically, FIG. 3 illustrates photographic diagrams of a metamaterial array including metalmaterials coated with samples containing peptides having similar sequences to a receptor binding protein (RBD) of each of SARS-CoV (A, C, E) and SARS-CoV-2 (B, D, and F).

FIG. 4A, FIG. 4B and FIG. 4C are diagrams illustrating a process for specifying the amino acid sequence of a coronavirus having an excellent absorption rate in a terahertz band in order to use a device for determining a coronavirus mutation or a device for diagnosing a coronavirus disease-19 according to an embodiment of the present invention.

FIG. 5A, FIG. 5B, FIG. 5C, FIG. 6A, FIG. 6B, FIG. 6C, FIG. 6D, FIG. 6E, FIG. 6F, FIG. 6G, FIG. 7A, FIG. 7B, FIG. 7C, FIG. 7D, FIG. 7E, FIG. 7F and FIG. 7G are diagrams illustrating determination results of coronavirus mutations using a device for determining a coronavirus mutation device or a device for diagnosing a coronavirus disease-19 according to an embodiment of the present invention.

### DETAILED DESCRIPTION OF THE INVENTION

Hereinafter, the present invention will be in detail explained with reference to the attached drawings. However, the present invention may be embodied in many different forms and, therefore, is not limited to the embodiments described herein. In addition, the accompanying drawings are provided only for a better understanding of the embodiments disclosed in the present specification and are not intended to limit technical ideas disclosed in the present specification. Therefore, it should be understood that the accompanying drawings include all modifications, equivalents and substitutions within the scope and sprit of the present invention. In order to clearly explain the present invention, parts irrelevant to the description have been omitted from the drawings, and the sizes and shapes of respective components shown in the drawings may be variously modified, and the same/like reference numerals refer to the same/like components throughout the specification.

The suffixes “module” and “unit” for components used in the description below are assigned or mixed in consideration of easiness in writing the specification and do not have distinctive meanings or roles by themselves. If it is determined that the detailed explanation on the well known technology related to the present invention makes the scope of the present invention not clear, the explanation will be avoided for the brevity of the description.

Throughout the specification, when one part is said to “include (have or provide)” any component, this does not exclude other components unless otherwise stated, but it means that one part may further “includes (have or provide)” other components.

Terms used in this specification are used to only describe specific exemplary embodiments and are not intended to restrict the present invention. An expression referencing a singular value additionally refers to a corresponding expression of the plural number, unless explicitly invited otherwise by the context. Components that are implemented in a distributed manner may be implemented in combination unless there are particular limitations. In this specification, terms, such as “comprise”, or “have”, are intended to designate those characteristics, numbers, steps, operations, components, or parts which are described in the specification, or any combination of them that exist, and it should be understood that they do not preclude the possibility of the existence or possible addition of one or more additional characteristics, numbers, steps, operations, components, or parts, or combinations thereof.

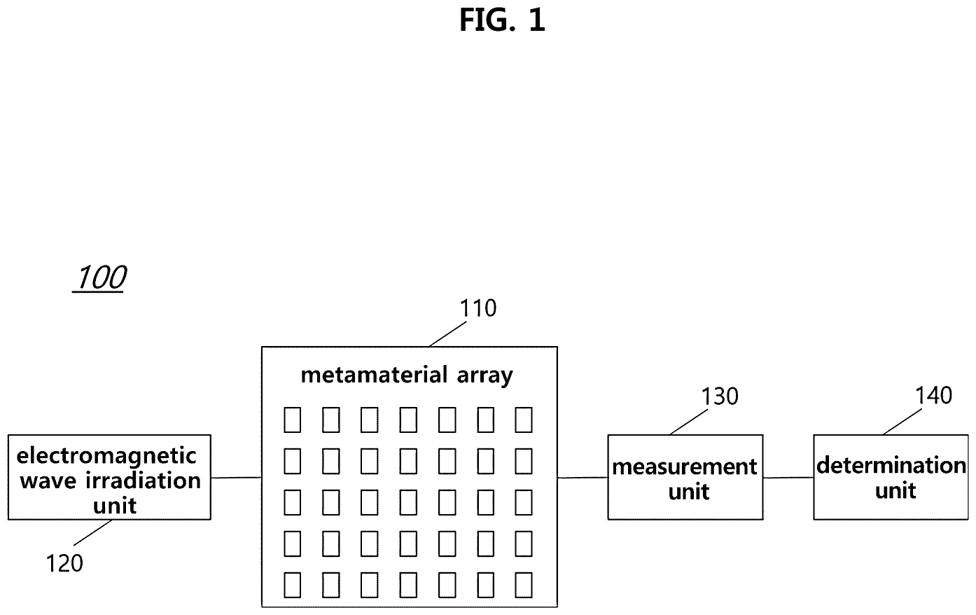

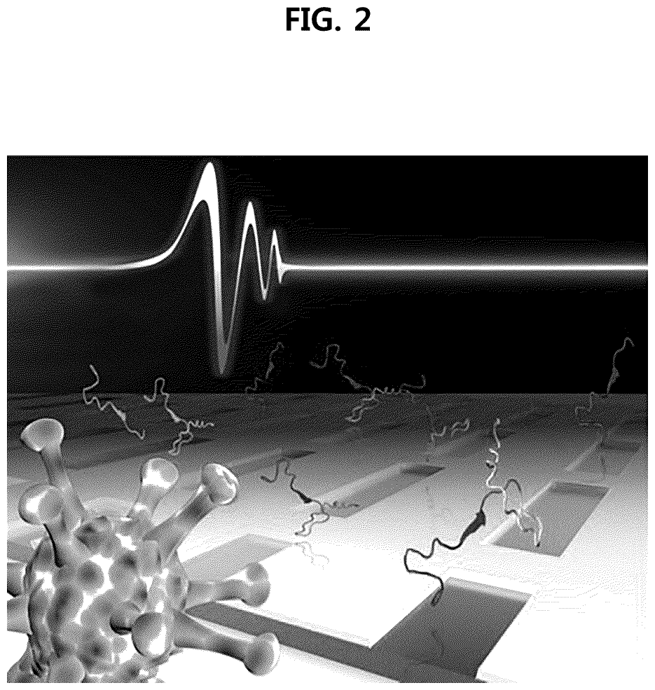

FIG. 1 is a block diagram illustrating a schematic configuration of a device for determining a coronavirus mutation 100 including a metamaterial array and an electromagnetic wave irradiation unit according to an embodiment of the present invention, and FIG. 2 is a diagram visually illustrating coronavirus determination in a sample and mutation determination using the device for determining a coronavirus mutation 100.

Prior to the detailed description, schematically describing the device according to one aspect of the present invention, the viruses of the coronavirus genus have a similar amino acid sequence of 82% or more, and the mutations are also diverse and new mutations are discovered in a short period of time. Therefore, it is urgent to develop a technology capable of quickly determining the mutations. The device according to one aspect of the present invention can sensitively measure a terahertz optical signal reflecting the unique information of a sample even in a small amount by using a metamaterial that amplifies a signal of a specific band of electromagnetic waves. Specifically, in a device according to one aspect of the present invention, after specifying amino acids having an excellent absorption rate in a terahertz band, a terahertz metamaterial to amplify the signal was developed. In addition, after uniformly dispersing the sample on the surface of the metamaterial, by analyzing the optical constant value of the sample, it was possible to detect monomers within a few minutes and to estimate the sample characteristics such as charge, polarity, and hydrophobicity index and sample amount, and mutant viruses having similar structures could be identified by detecting changes in amino acid units.

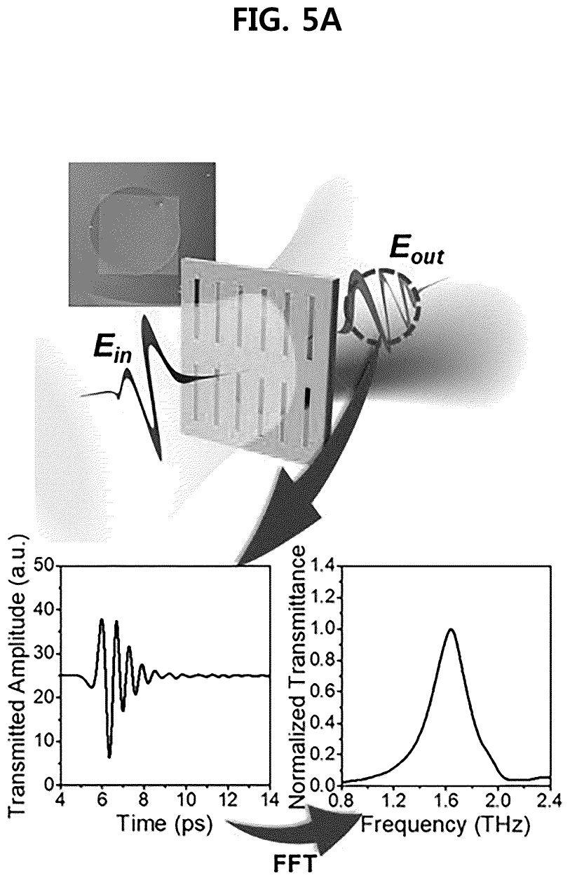

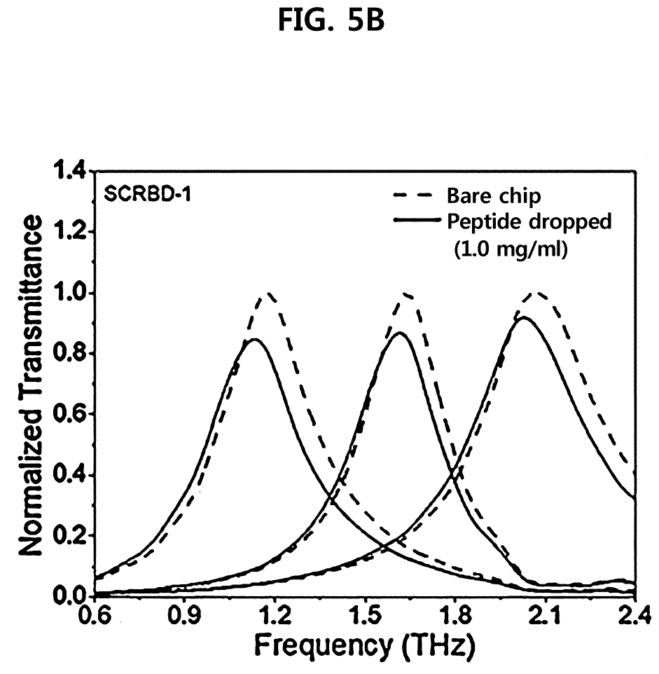

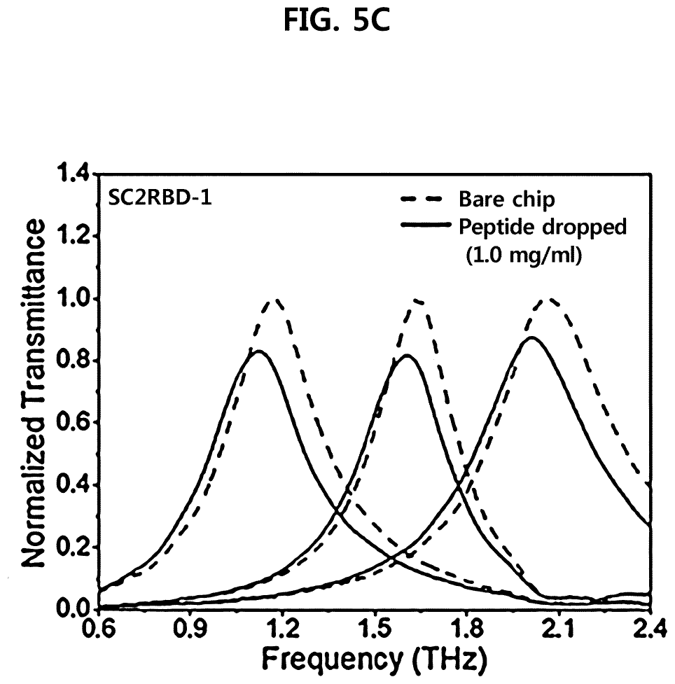

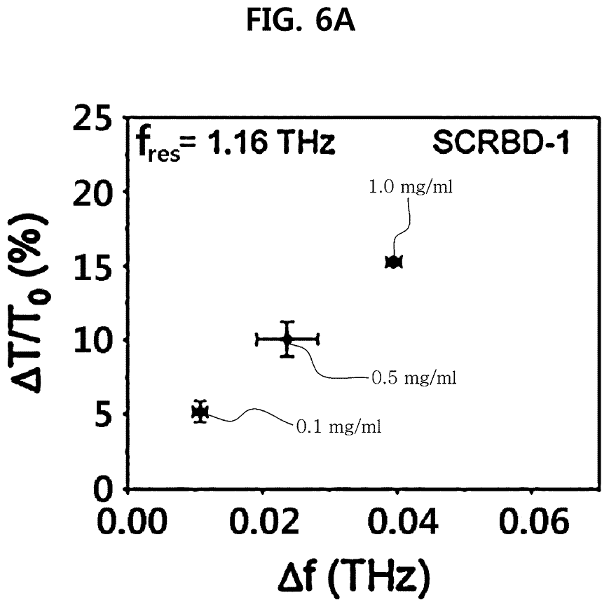

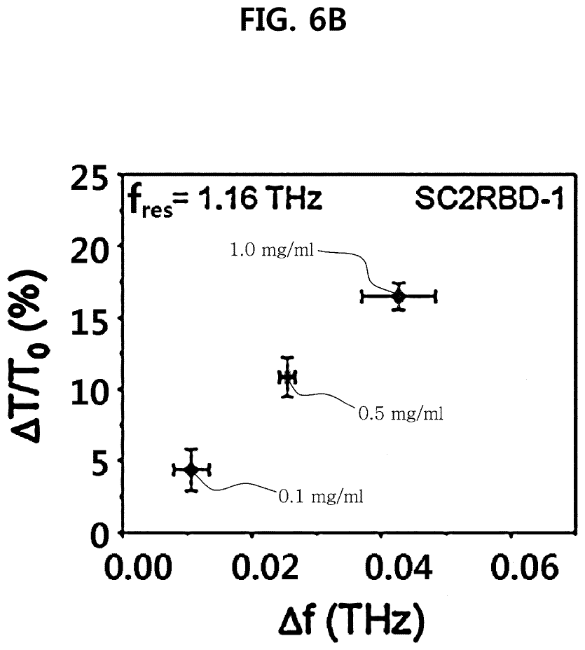

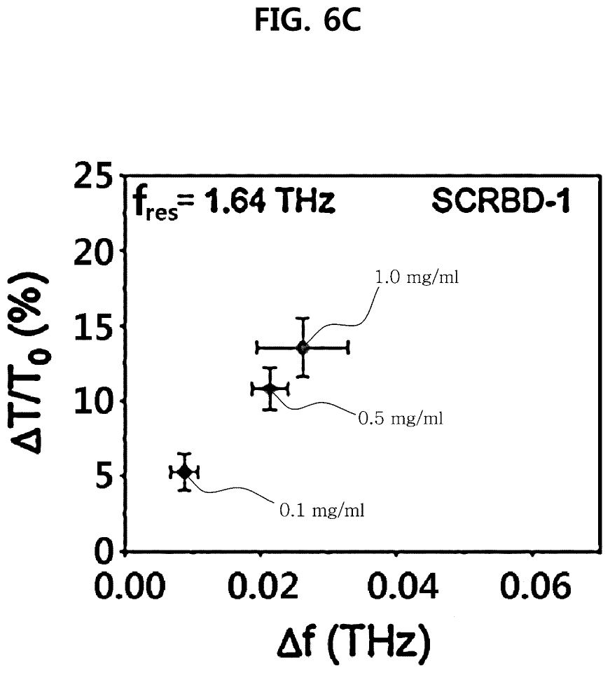

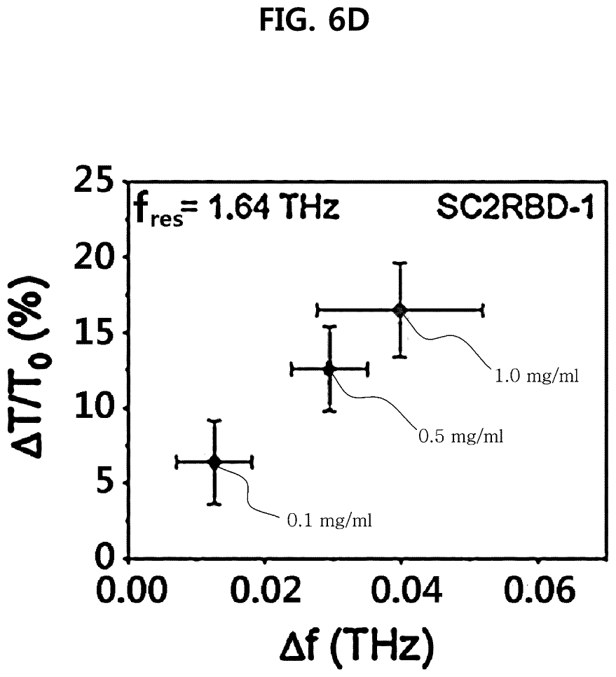

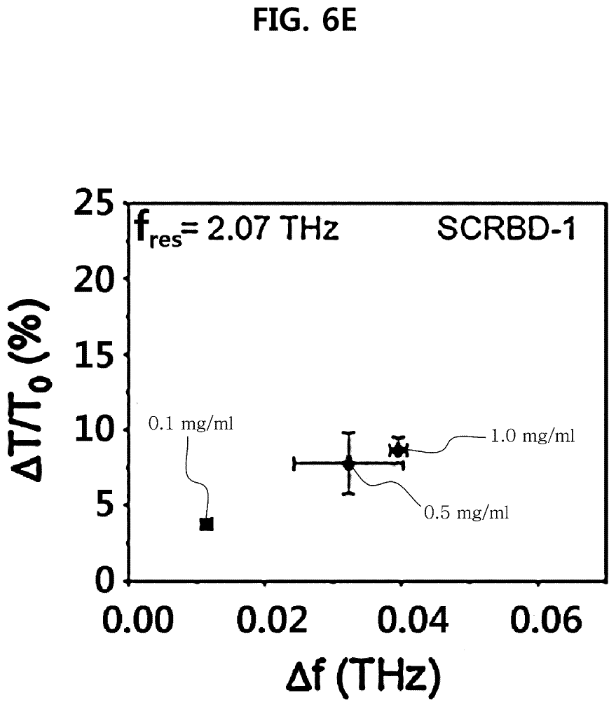

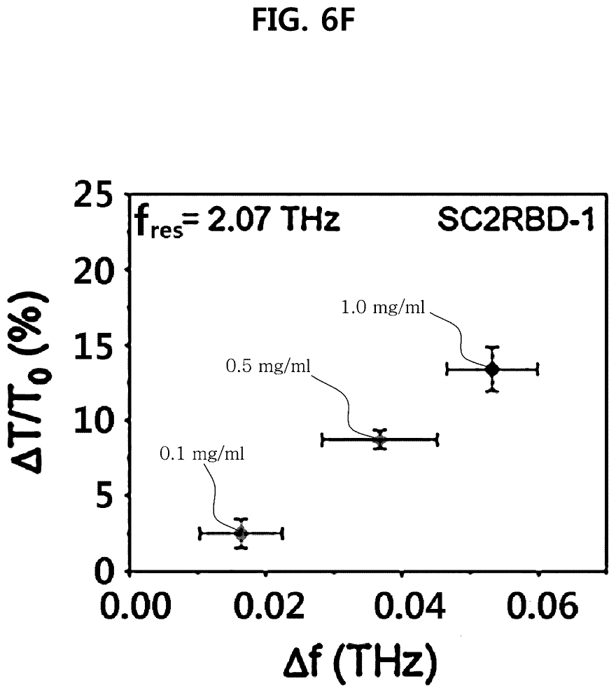

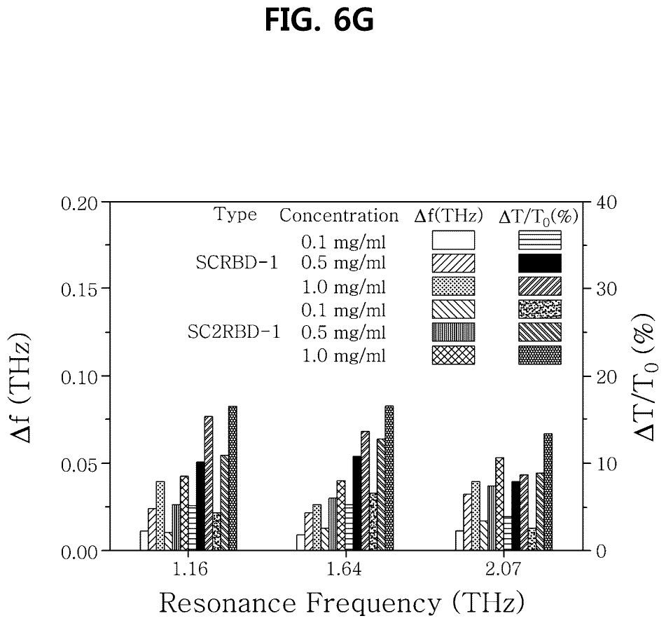

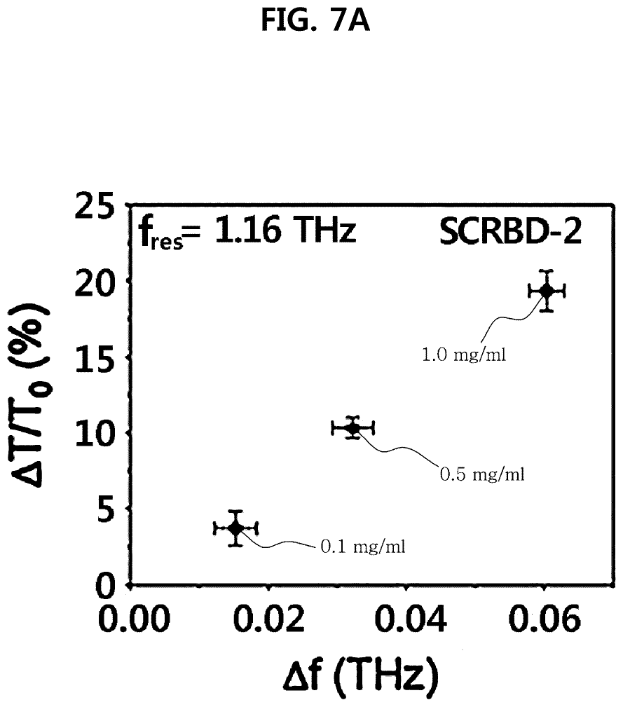

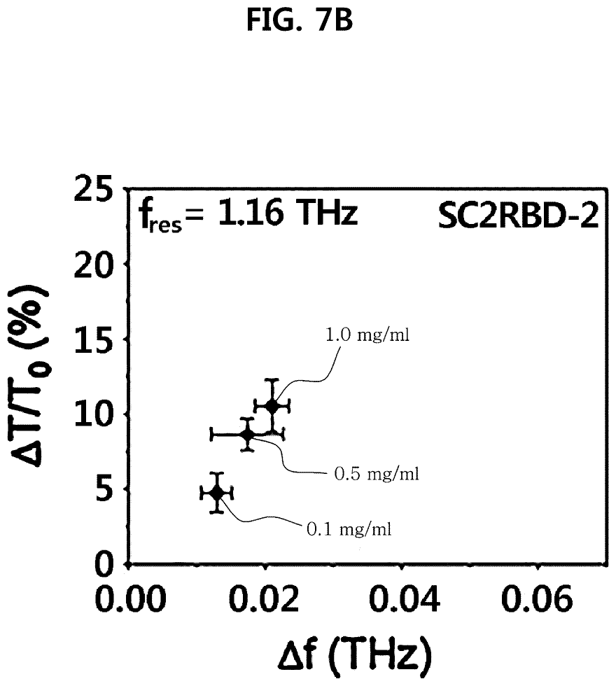

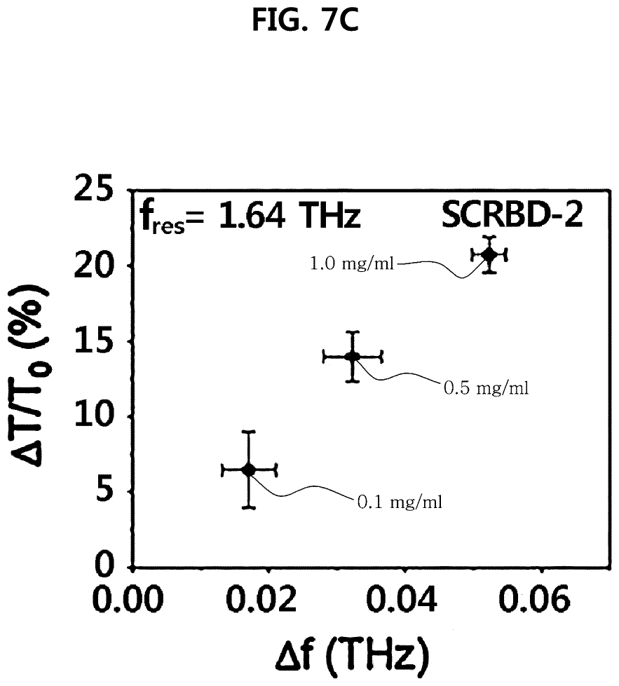

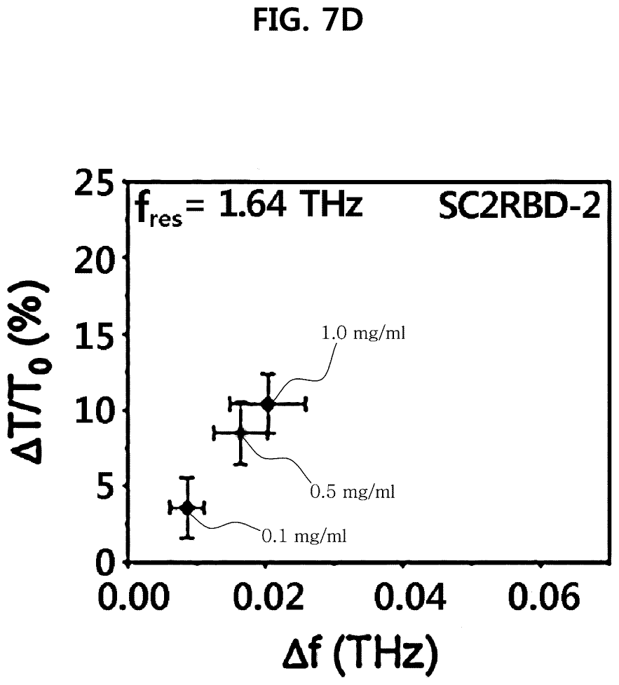

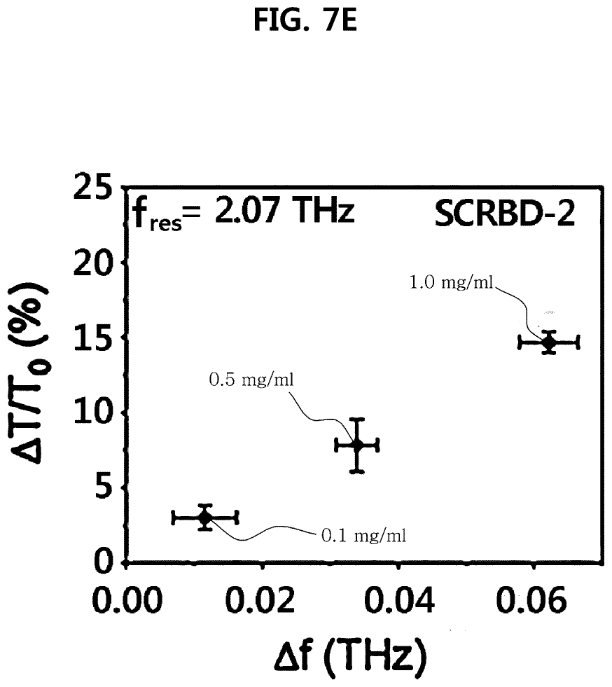

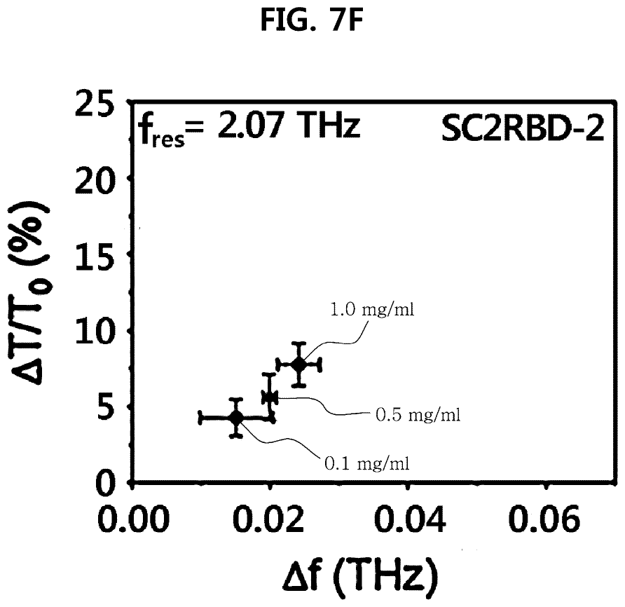

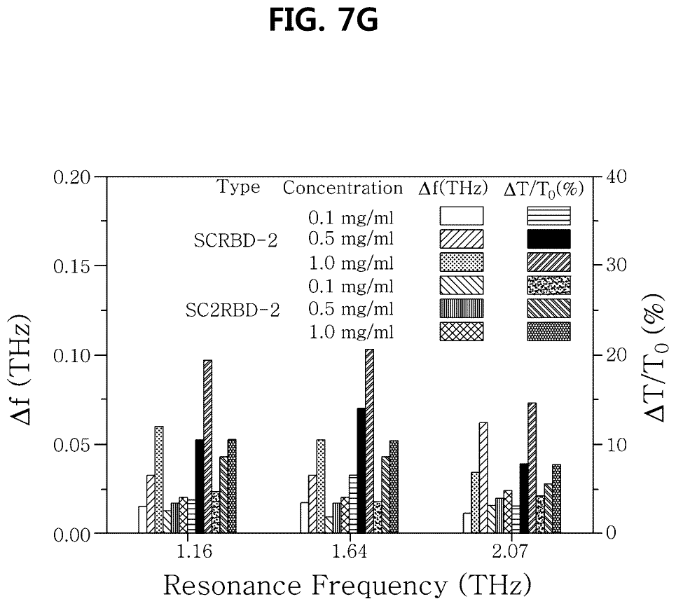

Referring to FIG. 1, the device 100 for determining a coronavirus mutation according to an aspect of the present invention may include a metamaterial array 110 and an electromagnetic wave irradiation unit 120, and the device may further include a measurement unit 130 that measures one or more measurement factors selected from the group consisting of a frequency shift and transmittance of the irradiated electromagnetic wave, and a determination unit 140 that determines the mutation of the coronavirus based on the measured measurement factors. Referring to FIG. 5A to 5C, a terahertz spectroscopy according to an aspect of the present invention is a method for analyzing the characteristics of a material based on the difference between an incident wave having a frequency in the terahertz band and a wave interacting with a material. This difference is known to be due to the optical properties of the material. The terahertz signal analysis may be largely represented by a change in transmittance change and a frequency shift. The change in transmittance corresponds to the absorption coefficient (or extinction coefficient) of the material, and the frequency shift is caused by the refractive index and the concentration of the detection factor collected in the active region. The device for determing a coronavirus mutation according to one aspect of the present invention is designed to be sensitive to the absorption rate of the material by configuring a terahertz metamaterial array having an intaglio structure and a transmissive terahertz spectroscopy system.

The metamaterial array 110 according to one aspect of the present invention includes a metamaterial, specifically, the metamaterial may be configured in the form of a unit cell 111, and the metamaterial array 110 may include a plurality of the unit cells.

Referring to FIG. 2 illustrated to explain the metamaterial array according to one aspect of the present invention, the metamaterial according to one aspect of the present invention is composed of asymmetric nanogaps 112 having constant intervals. The resonant frequency may be shifted by adjusting the length of the nanogap 112. In other words, the surface of the metamaterial array may include the nanogap 112 to amplify the electromagnetic waves of a specific frequency, and specifically, one or more selected from the group consisting of the length, width, and thickness of the nanogap may be adjusted such that resonance occurs at the frequency of the natural vibration mode of the coronavirus. In addition, it is possible to determine whether the metamaterial array 110 and the device 100 for determining a coronavirus mutation operate according to the incident polarization of the terahertz electromagnetic wave irradiated by the electromagnetic wave irradiation unit according to one aspect of the present invention.

In addition, the metamaterial array 110 according to one aspect of the present invention may amplify some amino acids constituting the coronavirus (Cov). The coronavirus may be a virus belonging to the genus coronavirus. Some amino acids constituting the coronavirus may be a part of the amino acid sequence constituting the receptor binding protein (RBD) of the spike (S) region of the coronavirus, and specifically, the some amino acids may include leucine and asparagine. According to one embodiment of the present invention, in order to recognize changes in amino acid units of a biological sample, two pairs of units consisting of 20 amino acids with high similarity in the receptor binding domain of two types of coronavirus belonging to the genus coronavirus, severe acute respiratory syndrome coronavirus (SARS-CoV) and severe acute respiratory syndrome coronavirus-2 (SARS-CoV-2), were prepared. Leucine and asparagine were specified as the some amino acids constituting the coronavirus with an excellent absorption rate in the terahertz band.

A sample containing a coronavirus may be uniformly applied to the surface of the metamaterial according to one aspect of the present invention. In order to closely observe the signal change caused by terahertz electromagnetic waves, a liquid sample, specifically a sample containing coronavirus, and more specifically, a biological sample separated from an object must be uniformly distributed over the same area on the surface of the metamaterial. Conventionally, when a solution-based sample is applied to a sensing platform such as a metamaterial array according to an aspect of the present invention, there is a problem in that a ring-shaped band known as a coffee ring is easily formed, which makes measurement and analysis difficult. According to one aspect of the present invention, the sample containing the coronavirus may be uniformly applied to the surface of the metamaterial through optimization of a surface treatment technique and a drying method.

Regarding the surface treatment technique according to one aspect of the present invention, it is generally difficult to locate the solvent containing the detection factor in the active region of the metamaterial array due to its hydrophobic nature due to gold (Au) and high surface tension formed in a narrow width of about 500 nm. However, when the surface treatment technique according to one aspect of the present invention is used, in order to infiltrate a solvent mainly composed of water into the active region, a local region where the array of metamaterials is located may be surface treated to have hydrophilic characteristics.

In addition, in relation to the drying method according to one aspect of the present invention, even if the solvent is uniformly dispersed on the surface, during the drying process, a polymerized film may be non-uniformly formed due to shear stress and modulus. As the drying method, there are natural drying, heat drying, and vacuum drying. In the case of natural drying, it is not suitable for use as a rapid diagnosis method because the dry is performed for a long time (more than 12 hours) in a dry atmosphere (relative humidity <2%). The heat drying is not appropriate because it may change the properties of biological samples. The vacuum drying method promotes evaporation of the solvent by lowering the pressure inside a chamber, and has the advantage of being able to perform the dry within a short time without deformation of the biomaterial. As a drying method according to one aspect of the present invention, the vacuum drying method is optimized so that coronavirus mutations can be determined using the metamaterial array.

That is, the device for determining a coronavirus mutation according to one aspect of the present invention can secure a terahertz signal with excellent reproducibility and reliability by applying the sample dispersion technique including the surface treatment technique and the drying method.

The electromagnetic wave irradiation unit 120 according to one aspect of the present invention may irradiate terahertz electromagnetic waves toward the metamaterial array 110.

The device for determining a coronavirus mutation 100 according to one aspect of the present invention constitutes a transmissive terahertz system with low light loss to improve the sensitivity of sample determination. The absorption rate of the coronavirus monomers present in the sample depends on the composition and ambient temperature, which is related to the change in the intensity of the terahertz signal. Therefore, since the frequency change of the signal is proportional to the refractive index and the number of molecules trapped inside the nanogap, it may be used for quantitative detection analysis.

According to one embodiment of the present invention, when a sample containing a coronavirus is uniformly dispersed on the surface of the metamaterial composed of gold and an amino acid present in the coronavirus has a positive charge, a signal change according to the amino acid composition ratio was clearly observed from a metamaterial having a resonant frequency matching the terahertz absorption band of a specific amino acid, whereas a signal change depending on the presence or absence of a sample was not significant when the resonant frequency did not match. On the other hand, when the amino acid present in the coronavirus has a negative charge, non-uniform dispersion is made due to the repulsive force with the metamaterial surface and the rapid drying, and the mismatch of the effective refractive index on the surface of the metamaterial dramatically limits the terahertz signal change. From the quantitative analysis results of all these monomers, a linear signal change versus concentration change was identified, and a detection limit of 41.7 μM was identified.

In another aspect, the present invention provides a method for determining a coronavirus mutation including the steps of applying a sample containing a coronavirus to the surface of the metamaterial of the device for determining a coronavirus mutation; and drying the metamaterial array including the metamaterial to which the sample is applied. The description of the metamaterial, coronavirus, sample, metamaterial array, sample application on the surface of the metamaterial, and drying of the metamaterial array in the device for determining a coronavirus mutation is also applied to this method for determining a coronavirus mutation.

A method for determining a coronavirus mutation according to an aspect of the present invention may include the step of applying a sample containing a coronavirus to the surface of a metamaterial of the device for determining a coronavirus mutation.

A method for determining a coronavirus mutation according to an aspect of the present invention may include the step of drying the metamaterial array including the metamaterial to which the sample is applied.

A method for determining a coronavirus mutation according to an aspect of the present invention may further include the step of determining a mutation of the coronavirus present in the sample by measuring one or more measurement factors selected from the group consisting of the frequency shift and transmittance of the electromagnetic wave irradiated by the device.

In another aspect, the present invention provides a device for diagnosing a coronavirus disease-19 (SARS-CoV-2) including a metamaterial array that includes a metamaterial and amplifies electromagnetic wave of a specific frequency; and an electromagnetic wave irradiation unit that irradiates the terahertz electromagnetic wave toward the metamaterial array, wherein the metamaterial array amplifies some amino acids constituting a coronavirus (Cov), and a biological sample separated from an object is uniformly applied to a surface of the metamaterial. The description of the metamaterial, coronavirus, sample, metamaterial array, and electromagnetic wave irradiation unit in the device for determining a coronavirus mutation is also applied to this device for diagnosing a coronavirus disease-19.

The coronavirus according to one aspect of the present invention may be a virus belonging to the genus coronavirus. Some amino acids constituting the coronavirus may be a part of the amino acid sequence constituting the receptor binding protein (RBD) of the spike (S) region of the coronavirus, and specifically, the some amino acids may include leucine and asparagine.

A device for diagnosing a coronavirus disease-19 according to an aspect of the present invention may further include a measurement unit that measures one or more measurement factors selected from the group consisting of a frequency shift and transmittance of the irradiated electromagnetic wave; and a determination unit that determines the mutation of the coronavirus based on the measured measurement factors, so that it may diagnose whether or not the object is infected with the coronavirus disease-19.

The metamaterial according to one aspect of the present invention may be composed of asymmetric nanogaps having constant intervals, and the resonant frequency may be shifted by adjusting the length of the nanogap. In other words, the surface of the metamaterial array may include the nanogap to amplify the electromagnetic waves of a specific frequency, and specifically, at least one or more selected from the group consisting of the length, width, and length of the nanogap may be adjusted to cause the resonance occurrance at the frequency of a natural vibration mode of the coronavirus.

Hereinafter, the present disclosure will be described in detail through examples and experimental examples. However, the following examples and experimental examples are for illustrative purposes only and it will be apparent to those of ordinary skill in the art that the scope of the present disclosure is not limited by the examples and experimental examples.

### Example 1

### Metamaterial Surface Treatment

In order to manufacture a device for determining a coronavirus mutation according to one aspect of the present invention, a metamaterial was treated on the surface of a metamaterial array in the following manner.

Specifically, a PDMS mask having an empty space having a diameter of about 3 mm was manufactured as a metamaterial array, and ozone treatment was performed on the surface of the metamaterial array to form a hydroxyl group (OH− group). Then, the PDMS mask was attached to the metamaterial and heat treated in an oven at about 60° C. for 30 minutes to form a conformal bond, and ozone treatment was performed once more to realize hydrophilicity an active region.

### Example 2

### Vacuum Drying

In order to dry the metamaterial array to which the metalmaterial was treated prepared in Example 1 without the biomaterial deformation, the following vacuum drying method was performed.

First, a solvent was applied to the hydrophilic treated metamaterial array of Example 1, and a corresponding specimen was moved into a chamber. Then, it was dried for 15 minutes using a vacuum pump connected to the chamber.

### Experimental Example

### Identification of Determination Effect of Each Coronavirus Mutation Having a Different Structure

The metamaterial array prepared according to Examples 1 and 2 has a specific resonance frequency. Experiments were conducted to identify whether respective different coronavirus mutations having different structures could be determeind using the metamaterial array prepared according to Examples 1 and 2 above.

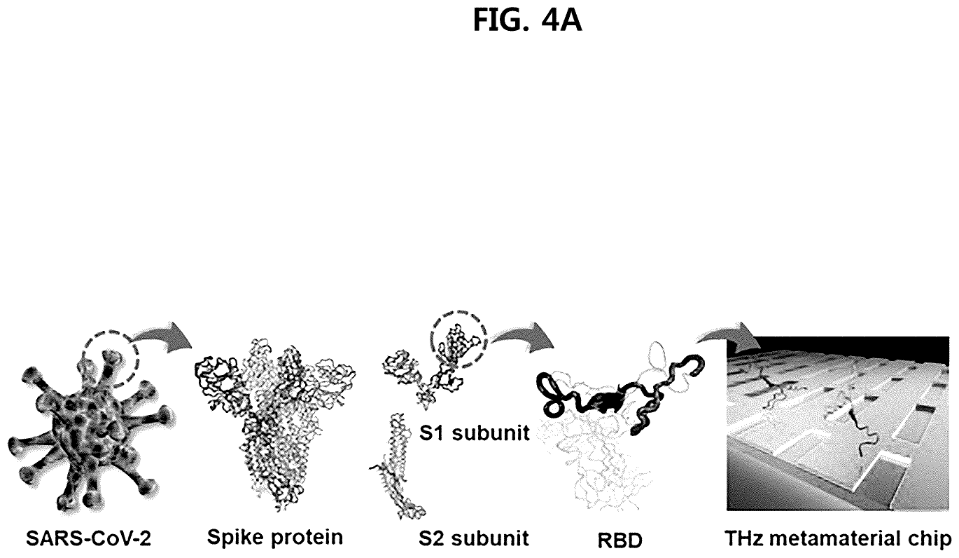

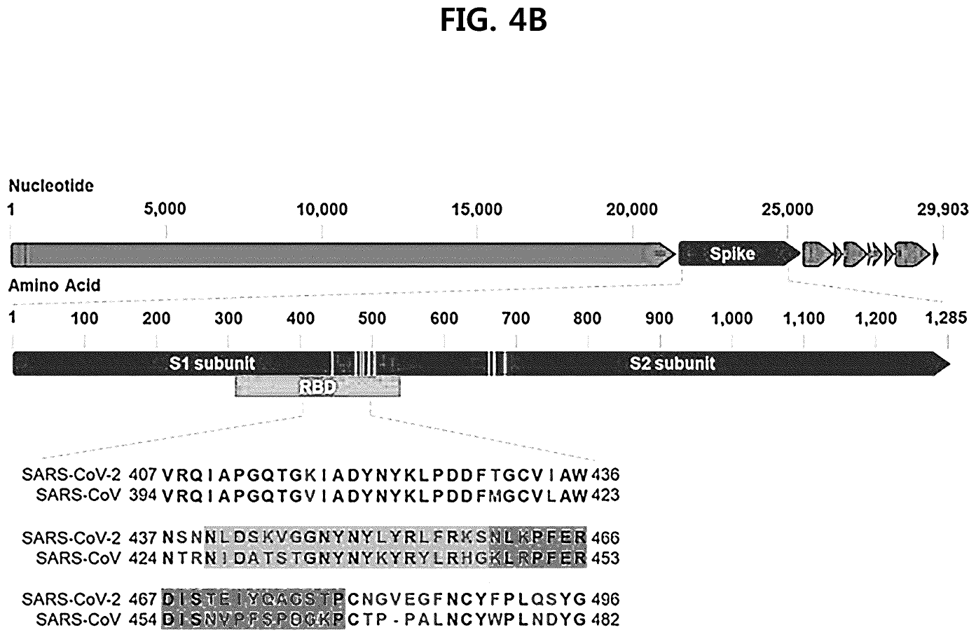

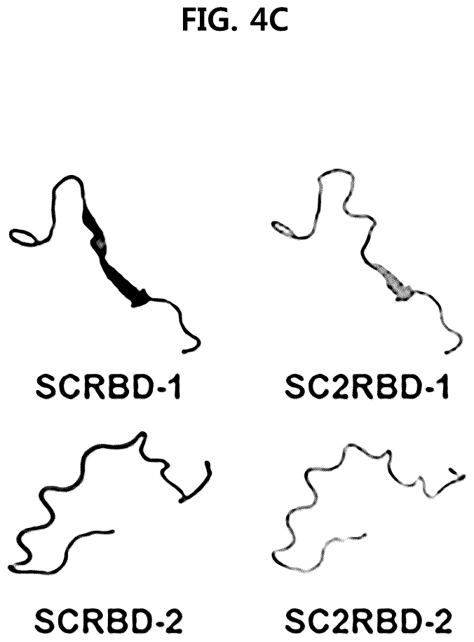

Specifically, three types of metamaterial arrays having frequencies of 1.16 THz, 1.64 THz, and 2.07 THz were prepared by the methods of Examples 1 and 2. In order to identify changes in amino acid units, two pairs of peptides with similar sequences were selected among receptor binding proteins (RBDs) that play an important role when SARS-CoV-2 and SARS-CoV penetrate the human body (FIG. 4A to 4C). The first pair has a structure of α-helix and β-strand, and is characterized by a different composition ratio of leucine and asparagine, which have high absorption rates in the terahertz region. The second pair has a κ-helix structure, and proline was selected as an analysis target.

In the first pair, leucine and asparagine have high absorption rates around 1.64 THz and 2.07 THz, but low absorption rates at 1.16 THz. Therefore, the difference in signal was clearly shown in the metamaterial arrays having resonance frequencies of 1.64 THz and 2.07 THz, respectively (FIG. 6A to 6G). The corresponding peptides have an isoelectric point (pI value) of about 10, so they showed positive polarity in distilled water with a pH of about 6.8, and as a result, they were uniformly dispersed on the surface of gold (Au), which showed negative polarity.

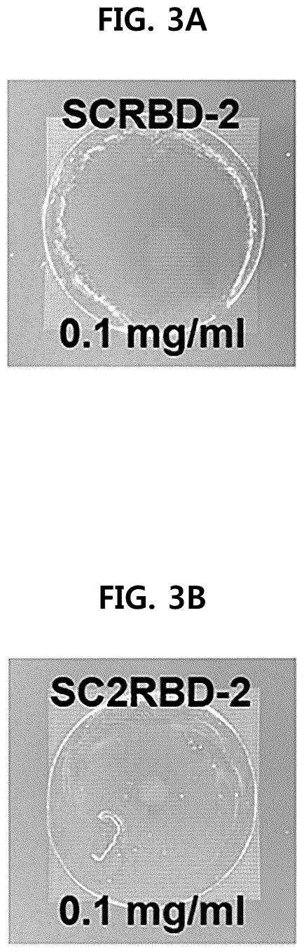

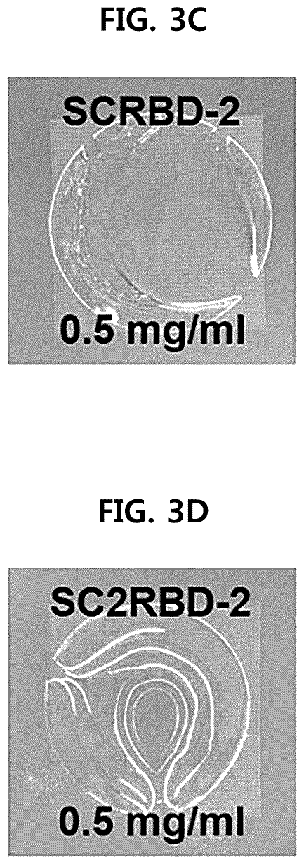

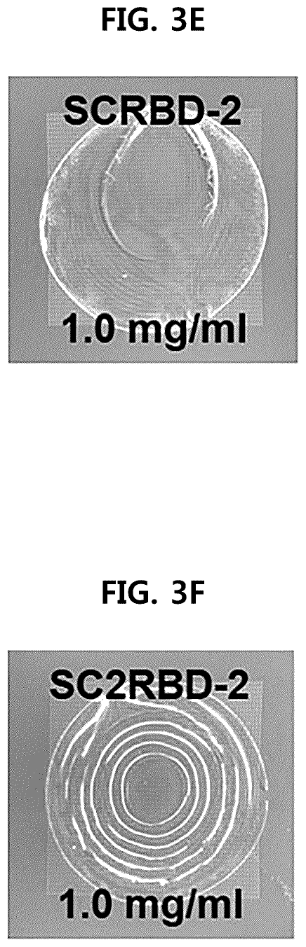

In the second pair, the signal of the SARS-CoV-2 peptide was detected to be much lower than that of the SARS-CoV peptide, regardless of the composition ratio and absorption rate of proline and the resonant frequency of the metamaterial array (FIG. 7A to 7G). The SARS-CoV peptide had a pI value of about 10, and, similarly to the previous samples, was uniformly dispersed on the surface of the metamaterial array (A, C, E in FIG. 3). On the other hand, the SARS-CoV-2 peptide with a pI value of about 4.4 had a negative polarity, resulting in a repulsive force with the surface of the metamaterial array, so that non-uniform dispersion (B, D, F in FIG. 3) and signal loss (B, D, F in FIG. 7) were occurred.

According to the above results, the changes in amino acid units of some of SARS-CoV and SARS-CoV-2 peptides were detected by analyzing the terahertz signal reflecting not only the optical constant value of the peptides but also the charge characteristics and hydropathy using the metamaterial array of Examples 1 and 2.

Through this, the metamaterial according to one aspect of the present invention can amplify the signal by specifying the amino acid of a virus belonging to the genus coronavirus, which has an excellent absorption rate in the terahertz band. The problem of forming a coffee ring that interferes with the conventional measurement and analysis of solution-based samples can be solved by applying a sample containing coronavirus to the surface of a metamaterial by the method according to one aspect of the present invention. Accordingly, using the device for determining a coronavirus mutation according to one aspect of the present invention, by analyzing the optical constant value of sample, it is possible to more quickly and accurately determine the coronavirus mutation, and furthermore, there is an excellent effect of diagnosing the coronavirus disease-19.

### DESCRIPTION OF REFERENCE NUMERALS

100: device for determining a coronavirus mutation

110: metamaterial array

111: unit cell

112: nanogap

120: electromagnetic wave irradiation unit

130: measurement unit

140: determination unit

### STATEMENT REGARDING PRIOR DISCLOSURES BY THE INVENTOR OR A JOINT INVENTOR

The inventors of the present application have made the following related disclosures: (1) Soo Hyun LEE et al., “Detection and discrimination of SARS-CoV-2 spike protein-derived peptides using THz metamaterials,” Biosensors and bioelectronics, Jan. 14, 2022, Vol. 202, No. 113981; (2) “Development of new technology to identify variants of COVID-19 with terahertz waves,” Yonhapnews, Feb. 22, 2022; (3) Jeongmin JANG et al., “Label-Free Terahertz Metamaterial Sensing on the SARS-CoV-2 variants,” The 21st Advanced Lasers and Their Application: ALTA 2022, May 2022; (4) Minah SEO, “Detection and discrimination of COVID-19 variants using terahertz metamaterial sensing,” NANO Korea 2022 (The 20th international Nanotech Symposium & Exhibition), July 2022; and (5) Jeongmin JANG et al., “Label-Free Detection and discrimination of SARS-CoV-2 Variants Using Terahertz Metamaterial Sensing Platform,” The 47th International Conference on Infrared, Millimeter and Terahertz Waves, August 2022. The related disclosures were made less than one year before the effective filing date (Aug. 5, 2022) of the present application. For the disclosures (2), (3), and (4), the inventors of the present application are the same as or include those of the related disclosures. For the remaining disclosures (1) and (5), the disclosure (1) includes two authors (Sang-Hun Lee and Jisung Kwak) and the disclosure (5) includes an author (Yeeun Roh), who are not included in the joint inventors of the present Application. However, these authors worked as an equipment operator and a technician and did not make contribution to conception of the invention, and thus are not included in the joint inventors of the present Application. Accordingly, the related disclosures are grace period inventor disclosures, and thus are disqualified from prior art under 35 U.S.C §102(a)(1) against the present application. See 35 U.S.C §102(b)(1)(A).

What is claimed is:

1. A device for determining a coronavirus mutation, comprising:
a metamaterial array that includes a metamaterial and amplifies electromagnetic wave of a specific frequency; and
an electromagnetic wave irradiation unit that irradiates terahertz electromagnetic wave toward the metamaterial array,
wherein the metamaterial array amplifies some amino acids constituting a coronavirus (Cov).

2. The device of claim 1, wherein the some amino acids are part of amino acid sequence constituting a receptor binding protein (RBD) of a spike (S) region of the coronavirus.

3. The device of claim 2, wherein the some amino acides include leucine and asparagine.

4. The device of claim 1, wherein a sample containing the coronavirus is uniformly applied to a surface of the metamaterial.

5. The device of claim 1, further comprising:
a measurement unit that measures one or more measurement factors selected from the group consisting of a frequency shift and a transmittance of the irradiated electromagnetic wave; and
a determination unit that determines a mutation of the coronavirus based on the measured measurement factor.

6. The device of claim 1, wherein a surface of the metamaterial array includes a nanogap to amplify the electromagnetic wave of a specific frequency.

7. The device of claim 6, wherein one or more selected from the group consisting of a length, width and thickness of the nanogap is adjusted so that resonance occurs at a frequency of a natural vibration mode of the coronavirus.

8. A device for diagnosing a coronavirus disease-19(SRAS-CoV-2), comprising:
a metamaterial array that includes a metamaterial and amplifies electromagnetic wave of a specific frequency; and
an electromagnetic wave irradiation unit that irradiates terahertz electromagnetic wave toward the metamaterial array,
wherein the metamaterial array amplifies some amino acids constituting a coronavirus (Cov), and
a biological sample separated from an object is uniformly applied to a surface of the metamaterial.

9. The device of claim 8, wherein the some amino acids are part of amino acid sequence constituting a receptor binding protein (RBD) of a spike (S) region of the coronavirus.

10. The device of claim 9, wherein the some amino acides include leucine and asparagine.

11. The device of claim 8, further comprising:
a measurement unit that measures one or more measurement factors selected from the group consisting of a frequency shift and a transmittance of the irradiated electromagnetic wave; and
a determination unit that determines a mutation of the coronavirus based on the measured measurement factor,
wherein the device for determining a coronavirus disease-19 diagnoses whether or not the object is infected with the coronavirus disease-19.

12. The device of claim 8, wherein a surface of the metamaterial array includes a nanogap to amplify the electromagnetic wave of a specific frequency.

13. The device of claim 12, wherein one or more selected from the group consisting of a length, width and thickness of the nanogap is adjusted so that resonance occurs at a frequency of a natural vibration mode of the coronavirus.
